(19) United States
(12) Patent Application Publication
(10) Pub. No.: US 2026/0230779 A1
(43) Pub. Date: Aug. 6, 2026

(54) SENSOR DATA COLLECTION METHOD AND SENSOR DEVICE

(71) Applicant: NTT, Inc., Tokyo (JP)

(72) Inventors: Akio Tokura, Tokyo (JP); Kenichi Matsunaga, Tokyo (JP); Kazuhiko Takagahara, Tokyo (JP)

(21) Appl. No.: 19/148,641

(22) PCT Filed: Jan. 18, 2023
(86) PCT No.: PCT/JP2023/001287

§ 371 Date: Jul. 16, 2025

(51) Int. Cl. H04W 4/38; A61B 5/00; G01P 1/12; H04W 56/00; H04W 76/25

(52) U.S. Cl. CPC H04W 4/38; A61B 5/0002; H04W 56/0055; H04W 76/25; G01P 1/127

(57) ABSTRACT

A sensor device is provided herein. The sensor device includes a sensor front end configured to output sensor data indicating measured physical amounts, a wireless communication circuit configured to communicate with a receiver, a memory for storing the sensor data, and a micro processing unit (MPU). The MPU is configured to refer to session information managed by the wireless communication circuit and determine whether the sensor device is under connection with the receiver. The MPU stops storing the sensor data in the memory and wirelessly transmits the sensor data to the receiver in real-time when under connection. The MPU stores the sensor data in the memory when communication is disconnected but not due to disconnection packet reception from the receiver.

### CROSS-REFERENCE TO RELATED APPLICATIONS

This application is a national phase entry of PCT Application No. PCT/JP2023/001287, filed on Jan. 18, 2023, which application is hereby incorporated herein by reference.

### TECHNICAL FIELD

The present invention relates to a sensor data collection method and a sensor device in which sensor data is wirelessly transmitted.

### BACKGROUND

In an IoT (Internet of Things) society where everything is connected to the Internet, it is expected that various sensors will be connected to a network to collect a wide variety and large amount of data and that the data will be analyzed to extract useful information for humans. When the sensor data acquired by the sensors is collected, data transfer by wireless is generally required in order to improve a degree of freedom of installation, and in particular, when collecting biological data, wireless communication further contributes to improve convenience (NPL 1).

Although wireless communication transmission of the sensor data is effective for improving convenience, there is a possibility that unexpected data missing may occur when a wireless environment suddenly deteriorates. As a technique for preventing such data missing, a method for storing all data on a sensor side (NPL 2), a method for performing delivery confirmation (chapter 3.8 in NPL 3), and the like are known.

However, since the method of storing all data in the sensor device requires a large-capacity memory to store a large amount of data, a problem of cost increase and size increase of the sensor device is likely to occur. On the other hand, although real-time data transmission by wireless and data backup by the sensor device may be performed in parallel, data duplication occurs in this case, and the convenience of data processing and analysis is reduced. In addition, in the method for performing the delivery confirmation, a stability of transmission and reception is increased, but there is a problem that communication speed is adversely affected by overhead caused by the delivery confirmation.

### CITATION LIST

### Non Patent Literature

[NPL 1] Nahoko Kasai, Takayuki Ogasawara, Hiroshi Nakashima, and Shingo Tsukada, “Development of Functional Textile ”hitoe“: Wearable Electrodes for Monitoring Human Vital Signals”, IEICE, Communications Society Magazine, vol. 11, no. 1, pp. 17-23, Jun. 1, 2017, Online ISSN 2186-0661, <https://doi.org/10.1587/bplus.11.17>

[NPL 2] “Holter recorder eMEMO WR-100”, medical equipment package insert, Fukuda Denshi Co., Ltd., November 2020, <https://www.pmda.go.jp/PmdaSearch/kikiDetail/ResultDataSetPDF/670053_228ADBZX00113000_A_02_01>

[NPL 3] RFC9293 Transmission Control Protocol (TCP), Internet Engineering Task Force (IETF), August 2022, <https://www.rfc-editor.org/rfc/rfc9293.html>

### SUMMARY

### Technical Problem

The present invention has been made for solving the above-described problem, and an object of the present invention is to provide a sensor data collection method and a sensor device capable of significantly suppressing sensor data missing and achieving low cost, stable, and automatic data collection.

### Solution to Problem

A sensor data collection method of the present invention is characterized in that it includes a first step of, by a sensor device, referring to session information managed by a wireless communication unit of the sensor device and determining whether or not to be under connection with a receiver, a second step of, by the sensor device, stopping storing sensor data output from a sensor front end of the sensor device in real-time in a memory of the sensor device and wirelessly transmitting the sensor data to the receiver when determining to be under connection with the receiver based on the session information, and a third step of, by the sensor device, storing the sensor data in the memory when determining that communication with the receiver is disconnected based on the session information and determining that the communication is not disconnected due to disconnection packet reception from the receiver.

In addition, a sensor device of the present invention is characterized in that it includes a sensor front end configured to output sensor data indicating measured physical amounts, a wireless communication unit configured to communicate with a receiver, a memory for storing the sensor data, and an MPU configured to stop storing the sensor data in the memory and wirelessly transmit the sensor data to the receiver in real-time via the wireless communication unit when determining to be under connection with the receiver based on session information managed by the wireless communication unit and store the sensor data in the memory when determining that communication with the receiver is disconnected based on the session information and determining that the communication is not disconnected due to disconnection packet reception from the receiver.

### Advantageous Effects of Invention

According to the present invention, it is possible to automatically switch a sensor data transmission operation in real-time, a memory storage operation of only data which cannot be wirelessly transmitted, and a measurement operation end of the sensor device by condition determination based on session information related to a wireless communication state. In the present invention, even if a state in which data transmission is disabled due to deterioration of a radio wave situation or the like occurs, the sensor data missing does not occur. In addition, since real-time data transmission and data backup by the sensor device are not performed in parallel, data duplication does not occur. Since the sensor data can be easily linked in the present invention, the convenience of data utilization can be improved. In addition, since it is not necessary to store all sensor data in the memory of the sensor device in the present invention, a large-capacity memory as in the prior art is not required, and the cost of the sensor device can be reduced. In addition, since a conventional method by the delivery confirmation is not used in the present invention, communication speed is not reduced by the delivery confirmation.

### BRIEF DESCRIPTION OF THE DRAWINGS

FIG. 1 is a block diagram showing a configuration of a sensor data collection system according to a first example of the present invention.

FIG. 2 is a block diagram showing a configuration example of a sensor front end of a sensor device according to the first example of the present invention.

FIG. 3A to FIG. 3B are diagrams showing an operation of a conventional sensor device.

FIG. 4A to FIG. 4B are diagrams for explaining an operation of the sensor device according to the first example of the present invention.

FIG. 5 is a flowchart for explaining an operation of the sensor device according to the first example of the present invention.

FIG. 6 is a flowchart for explaining an operation of a receiver according to the first example of the present invention.

FIG. 7 is a diagram for explaining effects of the first example of the present invention.

FIG. 8A to FIG. 8B are diagrams for explaining an operation of a sensor device according to a second example of the present invention.

FIG. 9 is a flowchart for explaining an operation of the sensor device according to the second example of the present invention.

FIG. 10 is a diagram for explaining effects of the second example of the present invention.

FIG. 11 is a block diagram showing a configuration of a sensor data collection system according to a third example of the present invention.

FIG. 12 is a flowchart for explaining an operation of a sensor device according to the third example of the present invention.

FIG. 13 is a flowchart for explaining an operation of the sensor device according to the third example of the present invention.

FIG. 14 is a flowchart for explaining an operation of a receiver according to the third example of the present invention.

FIG. 15 is a diagram for explaining an operation of a data alignment unit according to the third example of the present invention.

FIG. 16 is a block diagram showing a configuration example of a computer that realizes the sensor data collection system according to the first example to the third example of the present invention.

### DETAILED DESCRIPTION OF ILLUSTRATIVE EMBODIMENTS

### Principles of the Invention

In a wireless sensor device that wirelessly transmits sensor data in real-time, it is not realized to prevent data missing caused by communication blockage due to deterioration of a wireless situation or distance to the receiver that has moved beyond a communication range because of limitation of improving transmission and reception performance of radio wave alone.

In order to fundamentally solve the problem of the data missing, the only way is that data which cannot be transmitted is stored in the sensor device and read out later. As mentioned above, when the data is always backed up in the memory, large memory capacity is required. In addition, since the data transmitted to the receiver and the data stored in the memory are independent from each other, the data duplication occurs and the data utilization is inconvenient.

In order to efficiently store data in the memory without deteriorating convenience, it is necessary to switch an operation from wireless data transmission to data storage in an internal memory at timing when data cannot be transmitted. However, since the communication with the receiver is blocked, it is impossible to adopt a method of sending an instruction from the receiver to switch the operation.

In a wireless communication standard for a sensor such as a BLE (Bluetooth (registered trademark) Low Energy), session information for confirming whether or not communication is continuously established (connected) is defined. That is, when there is the session information, it suggests that the wireless communication is performed smoothly, and when there is conversely no session information, it suggests that the wireless communication is disconnected. Therefore, by taking the session information into the determination of the operation, communication maintenance and communication blockage are separated, and by storing data scheduled to be transmitted in the memory of the sensor device only at the time of communication blockage and reading the sensor data later, a probability of occurrence of data missing can be reduced.

However, the session information is generally binary information indicating presence or absence of communication continuity. Therefore, it is impossible to distinguish a situation in which the user of the sensor data collection system intentionally completes or disconnects the communication from a situation in which the communication is disconnected due to deterioration of a wireless environment or the like without intention of the user based on the session information. Therefore, a case occurs, in which data is stored in the memory of the sensor device even after the user intentionally disconnects the communication. When data storage in the memory of the sensor device is continued, there is a possibility that the memory is tight and the operation of the sensor device fails.

Therefore, in the present invention, ternary information for the determination can be obtained by including a case classification due to a generation cause of a session information change (loss), and the situation in which the user intentionally completes or disconnects the communication and the situation in which the communication is unintentionally disconnected can be distinguished. Specifically, it is used to usually transmit a disconnection packet to the sensor device by disconnect processing when the user completes and disconnects the communication (measurement).

Loss of session information caused by a disconnection packet by the disconnect processing and loss of session information caused by reasons other than the disconnect processing can be clearly identified. Therefore, only the disconnection by the disconnect processing is used as determination condition of normal end of the measurement. Most of the loss of session information generated by reasons other than the disconnect processing occurs due to the connection confirmation timeout (Supervision timeout). A loss state of the session information caused by reasons other than the disconnect processing is determined to be a disconnection unintended by the user, and the operation is switched to store the data in the memory.

A function of automatically switching real-time transmission, memory storage, and measurement end of the sensor data can be realized by adopting the operation determination using the ternary information in the sensor device.

Note that, strictly speaking, the data missing may occur only for the internal determination time until the Supervision timeout occurs. However, it is sufficiently possible to suppress the data missing time to a practically acceptable level by appropriately adjusting the Supervision timeout value, or by buffering the data in the internal memory for a few seconds in consideration of the Supervision timeout determination time.

When the data stored in the memory of the sensor device is read out, it is desirable that additional processing by the user is not required. The reason is that forcing the user to frequently confirm and control the session state of the device and data accumulation in the memory reduces convenience for the user, and the feature of the sensor data collection system that allows automatic data collection during measurement is lost.

Therefore, it is desirable to automatically read the data stored in the memory of the sensor device by the wireless communication during the measurement. For this purpose, it is necessary to switch between a state in which the real-time data and the data stored in the memory are simultaneously transmitted in parallel when the data is stored in the memory of the sensor device and a state in which only the real-time data is transmitted when the data is not stored in the memory.

Normally, such switching of the transmission state is executed by setting command transmission from the receiver which is on a master side. However, if the sensor device is not designed and mounted so that both the transmission state of only real-time data and the simultaneous parallel transmission state of the real-time data and the data stored in the memory can be realized, the switching does not operate normally.

In addition, in the conventional backup method for always storing the sensor data in the memory, it cannot be realized that memory data is read during the measurement unless a performance capable of simultaneously performing two types of memory access processing of writing and reading to and from the memory is present. Such memory access processing is difficult to be realized by most of the wireless sensor devices having many restrictions on hardware.

On the other hand, in the present invention, since the data storage to the memory of the sensor device is not executed when the wireless communication with the receiver is continued, it is only necessary to simultaneously transmit the real-time data during the measurement and read the data from the memory, and this can be achieved even with the sensor device that has many hardware restrictions.

The sensor device of the present invention has a function of appropriately switching between the real-time data transmission and the data storage in the memory. The real-time data and the data stored in the memory are in complementary relationship to each other and do not duplicate each other, so that they can be easily combined and the convenience of data utilization is high.

When the time point information of the real-time data and the time point information of the data stored in the memory are matched, it is possible to know which data is combined to form a complete data set, and this further improves the utilization level of the data. In many cases, the sensor device has a real-time clock for holding the time point information by itself. The time point information between data can be matched by utilizing the real-time clock and a session information parameter (cumulative session information) reflecting the session information of the wireless communication, and data combination can be easily performed.

The cumulative session information indicates what number of times of connection have been made from the start time (first connection time between the sensor device and the receiver) of the measurement, for example. The cumulative session information shows the data stored in the memory after how many times the real-time data has been stored. The cumulative session information and the time point information are associated and managed as a data set, and the data set is added to a header part of the sensor data and stored it in the memory together with the sensor data, for example. By doing this, it is possible to specify the data stored in the memory is stored after how many times the real-time data has been made and when the storage is started.

In addition, the sensor device adds the time point information acquired from the real-time clock to the data stored in the memory. On the other hand, the real-time data is transmitted from the sensor device to the receiver and stored as a data file on the receiver side. The receiver adds the time point information acquired from its own real-time clock to the received data. In addition, when the communication is blocked and the data is disconnected, a data file update on the receiver side is stopped at that time point.

Therefore, if the time point synchronization can be realized with a certain degree of accuracy in the real-time clocks of the sensor device and the receiver, respectively, it can be seen that the data stored in the memory of the sensor device at substantially the same time point as the final update time of the real-time data may be combined to the rear end of the real-time data stored in the receiver side. By doing this, the time stamps of the real-time data and the data stored in the memory of the sensor device can be matched.

Since the real-time clock usually has clock accuracy equal to or higher than that of a crystal oscillator, they are unlikely to cause large time point deviations in a short period of time. However, when the time point is not matched even once after shipment from the factory of the sensor device and the receiver, and when there is no update of the time point for a long period of time, the time point deviations occur. Therefore, it is desirable to perform time point synchronization between the real-time clocks of the receiver and the sensor device by transmitting the time point information to the sensor device by the wireless communication after updating the real-time clock of the receiver side. Therefore, the time point synchronization of two types of data and the smooth combination between the data can be realized by setting the time point synchronization at the start of communication and at the restart of communication, and by synchronizing the time point of the sensor device and the receiver periodically in addition to the setting, for example.

### First Example

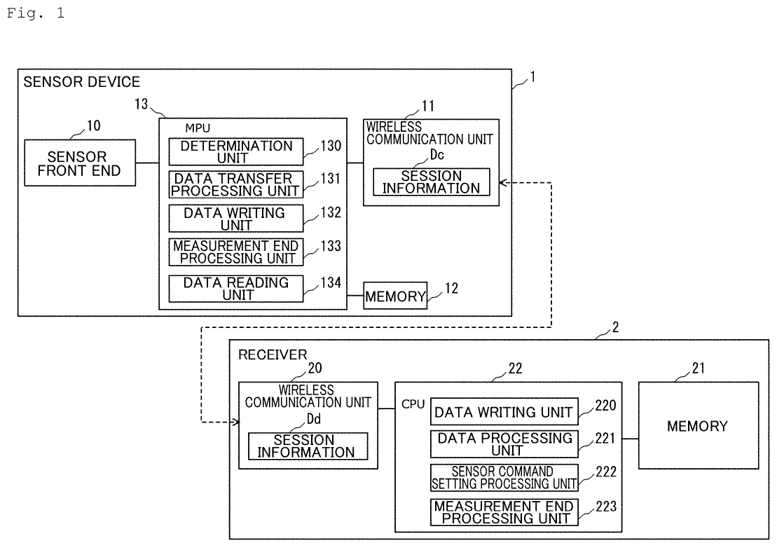

Hereinafter, the sensor device according to the example of the present invention will be described with specific examples. FIG. 1 is a block diagram showing a configuration of a sensor data collection system according to a first example of the present invention. The sensor data collection system is configured by a sensor device 1 that wirelessly transmits sensor data indicating measured physical amounts, and a receiver 2 that receives the sensor data.

The sensor device 1 includes a sensor front end 10 that outputs the sensor data including information on the measured physical amounts, a wireless communication unit 11 that communication with the receiver 2, a memory 12 that stores data and a program, and an MPU (Micro Processing Unit) 13 that controls the whole sensor device.

The MPU 13 executes processing in accordance with the program stored in the memory 12, and functions as a determination unit 130, a data transfer processing unit 131, a data writing unit 132, a measurement end processing unit 133, and a data reading unit 134.

The receiver 2 includes a wireless communication unit 20 that communicates with the sensor device 1, a memory 21 that stores data and a program, and a CPU (Central Processing Unit) 22 that controls the whole receiver.

The CPU 22 executes processing in accordance with the program stored in the memory 21, and functions as a data writing unit 220, a data processing unit 221, a sensor command setting processing unit 222, and a measurement end processing unit 223.

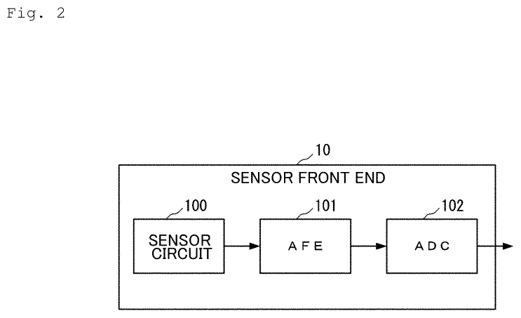

FIG. 2 is a block diagram showing a configuration example of the sensor front end 10 of the sensor device 1. The sensor front end 10 is configured by a sensor circuit 100 that measures physical amounts such as acceleration, angular acceleration, and electrocardiogram waveform, an analog front end circuit (Analog Front End, hereinafter referred to as AFE) 101 that performs processing such as amplification and noise removal of an analog signal output from the sensor circuit 100, and an AD converter (Analog to Digital Converter, hereinafter referred to as ADC) 102 that converts the analog signal output from the AFE 101 into digital data and outputs the digital data, for example. Note that the AFE 101 may contain the ADC.

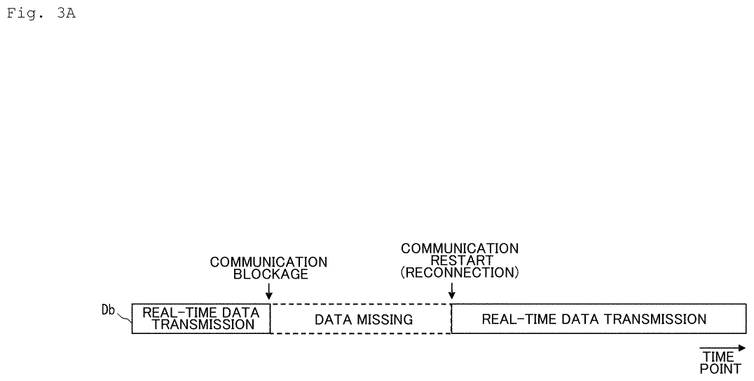

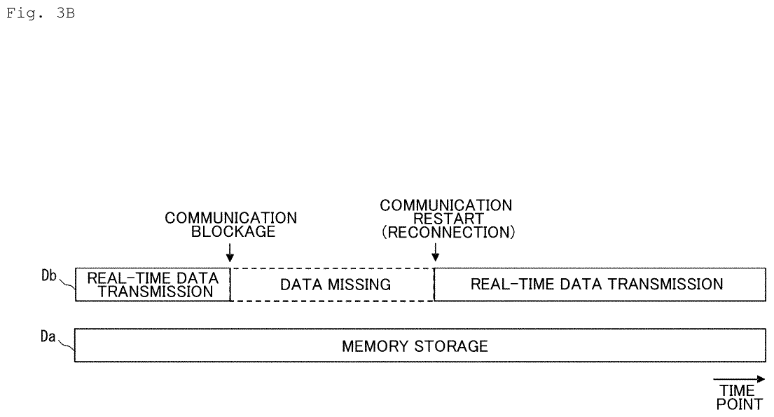

In a conventionally known operation, the sensor device 1 wirelessly transmits the sensor data Da output from the sensor front end 10 to the receiver 2 in real-time. When the sensor device 1 does not includes the memory, the real-time data Db (sensor data Da) cannot be transmitted at a point in time when communication with the receiver 2 is blocked as shown in FIG. 3A, and missing occurs in the data Db.

On the other hand, when the sensor device 1 includes the memory, the sensor data Da is stored in the memory independently of the real-time data Db transmitted to the receiver 2 as shown in FIG. 3B. The operations shown in FIG. 3A and FIG. 3B are not desirable operations in view of acquiring and utilizing the data set for a long period of time.

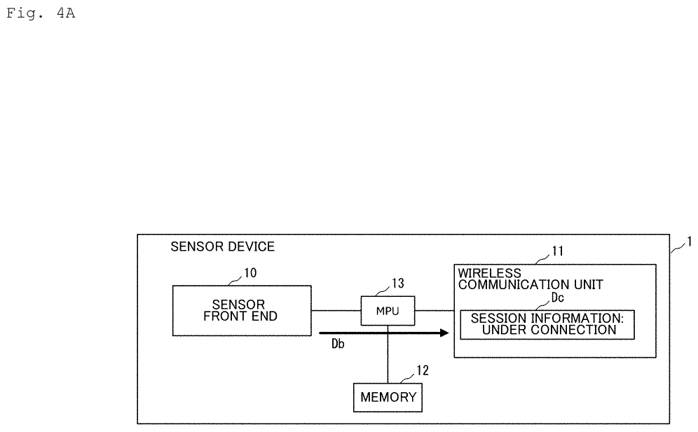

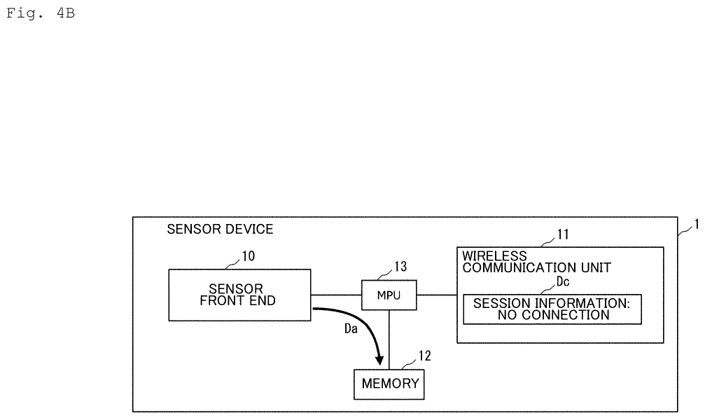

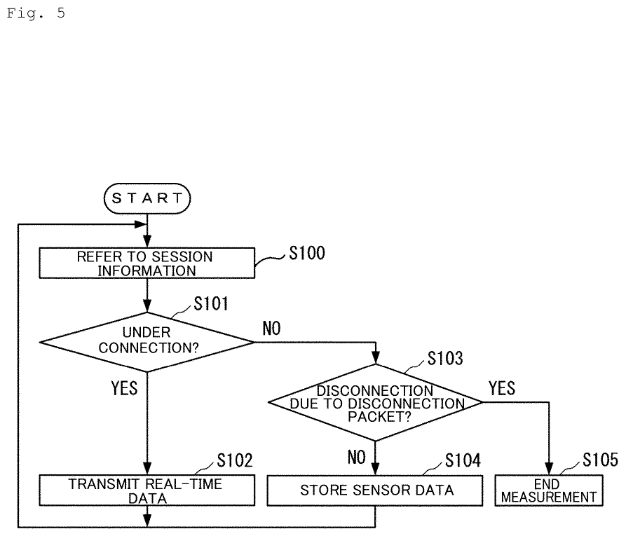

FIG. 4A and FIG. 4B are diagrams for explaining the operation of the sensor device 1 of the present example, and FIG. 5 is a flowchart for explaining the operation of the sensor device 1 of the present example.

The determination unit 130 of the sensor device 1 refers to session information Dc managed by the wireless communication unit 11 (step S100 in FIG. 5), and determines whether or not the sensor device 1 is under connection with the receiver 2 (step S101 in FIG. 5).

When the determination unit 130 determines to be under connection with the receiver 2 (YES in step S101), the data transfer processing unit 131 of the sensor device 1 stores the real-time data Db (sensor data Da acquired from the sensor front end 10) in a packet and passes the packet to the wireless communication unit 11 (FIG. 4A). The wireless communication unit 11 wirelessly transmits the packet received from the data transfer processing unit 131 to the receiver 2 under connection (step S102 in FIG. 5).

For example, in the wireless communication standard such as BLE, the receiver 2 which is master equipment periodically transmits an empty packet even when there is no content to be transmitted. The wireless communication unit 11 of the sensor device 1 which is slave equipment performs communication disconnection processing when the packet from the receiver 2 under connection cannot be received for a predetermined time (Supervision time) or longer. In this case, the session information Dc is changed to a value indicating no connection.

In addition, when the user of the sensor data collection system intentionally ends the communication (measurement), a disconnection packet is transmitted to the sensor device 1 by disconnect processing on the receiver 2 side. When receiving the disconnection packet from the receiver 2, the wireless communication unit 11 performs the communication disconnection processing.

When the determination unit 130 determines that the communication with the receiver 2 is disconnected (NO in step S101) and the determination unit 130 determines that the communication is not disconnected by disconnection packet reception from the receiver 2 (NO in step S103 in FIG. 5), the data writing unit 132 of the sensor device 1 stores the sensor data Da in the memory 12 as shown in FIG. 4B (step S104 in FIG. 5).

When the determination unit 130 determines that the communication with the receiver 2 is disconnected (NO in step S101) and the determination unit 130 determines that the communication is disconnected due to the disconnection packet reception from the receiver 2 (YES in step S103), the measurement end processing unit 133 of the sensor device 1 sets the sensor device 1 to a measurement operation end state (step S105 in FIG. 5). In the measurement operation end state, transmission of real-time data Db to the receiver 2 and storage of sensor data Da in the memory 12 are not performed, but packet reception from the receiver 2 is possible.

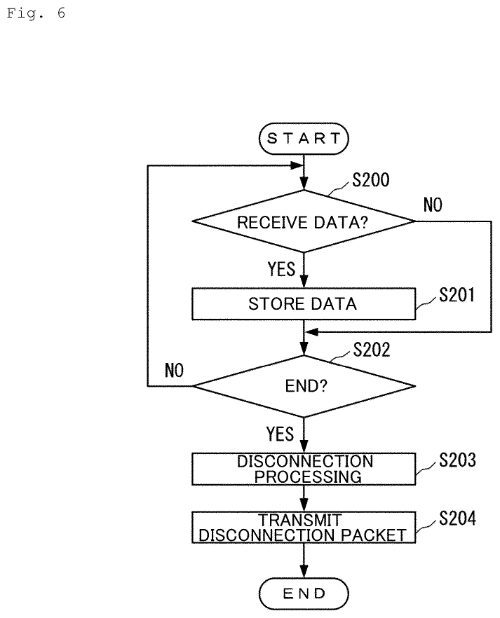

FIG. 6 is a flowchart for explaining an operation of the receiver 2. When receiving the data packet in which the real-time data Db is stored from the sensor device 1 (YES in step S200 in FIG. 6), the wireless communication unit 20 of the receiver 2 passes the received data packet to the data writing unit 220. The data writing unit 220 takes out the real-time data Db from the data packet and stores it in the memory 21 (step S201 in FIG. 6).

The data stored in the memory 21 is processed by the data processing unit 221. As the processing in this case, arithmetic processing, data transfer to a host device, and the like are included, for example. However, in the present invention, the data processing unit 221 is not an essential component.

Although not clearly shown in FIG. 6, the wireless communication unit 20 of the receiver 2 which is the master equipment periodically transmits the empty packet to the sensor device 1 under connection even if there is no content to be transmitted. On the other hand, the sensor device 1 which is the slave device returns a response packet. When the packet from the sensor device 1 under connection cannot be received for a predetermined time (Supervision time) or longer, the wireless communication unit 20 of the receiver 2 performs the communication disconnection processing. In this case, the session information Dd is changed to a value indicating no connection.

In addition, when the measurement operation is ended (YES in step S202 in FIG. 6), the measurement end processing unit 223 of the receiver 2 requests the wireless communication unit 20 to perform the communication disconnection processing. The wireless communication unit 20 performs the communication disconnection processing (step S203 in FIG. 6), and transmits the disconnection packet to the sensor device 1 (step S204 in FIG. 6).

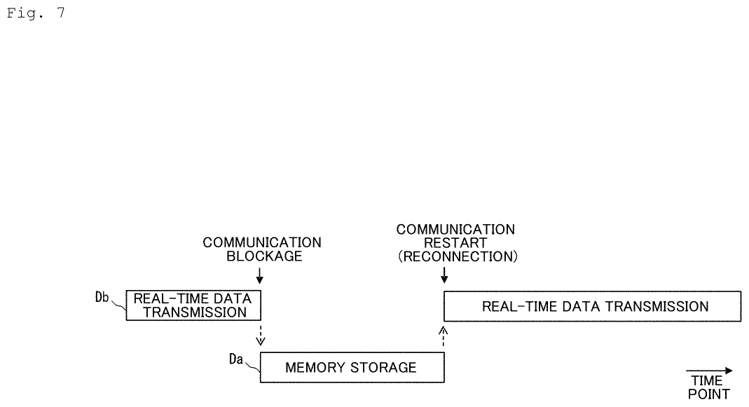

As described above, in the present example, three types of operations (data transmission, data storage, and measurement operation end) of the sensor device 1 can be automatically switched. Similarly to the prior art, the sensor device 1 cannot transmit the real-time data Db at a point of time when the communication with the receiver 2 is blocked, but automatically stores the sensor data Da in the memory 12 as shown in FIG. 7. The CPU 22 of the receiver 2 tries to connect with the sensor device 1 in the case of unintended disconnection (disconnection by Supervision timeout) by the user. When the sensor device 1 and the receiver 2 are reconnected and the communication is resumed, the operation of the sensor device 1 is switched and the real-time data Db is transmitted to the receiver 2.

In the present example, even if data transmission is disabled due to deterioration of the radio wave condition or the like, no missing occurs in the sensor data. In addition, since the real-time data transmission and the data backup by the sensor device 1 are not performed in parallel, data duplication is not generated. In the present example, the sensor data can be easily complemented (linked), so that the convenience of data utilization can be improved. In addition, in the present example, since it is not necessary to store all sensor data in the memory 12, the large-capacity memory as in the prior art is not required, and the cost of the sensor device 1 can be reduced. Further, in the present example, since the conventional method based on the delivery confirmation is not used, the communication speed does not decrease due to the delivery confirmation.

Note that the data stored in the memory 12 of the sensor device 1 can be read after the storage end. When reading the memory storage data, the sensor command setting processing unit 222 of the receiver 2 requests the wireless communication unit 20 to transmit a read request command. The wireless communication unit 20 transmits the read request command to the sensor device 1.

When the wireless communication unit 11 receives the read request command from the receiver 2, the data reading unit 134 of the sensor device 1 reads the sensor data Da stored in the memory 12, stores it in the packet, and passes it to the wireless communication unit 11. The wireless communication unit 11 wirelessly transmits the packet received from the data reading unit 134 to the receiver 2 under connection.

### Second Example

Next, a second example of the present invention will be described. The present example is an example of a more convenient way to acquire sensor data with respect to the first example. Also, in the present example, since the configuration of the sensor data collection system is similar to that of the first example, the description will be made with reference to the reference numerals in FIG. 1.

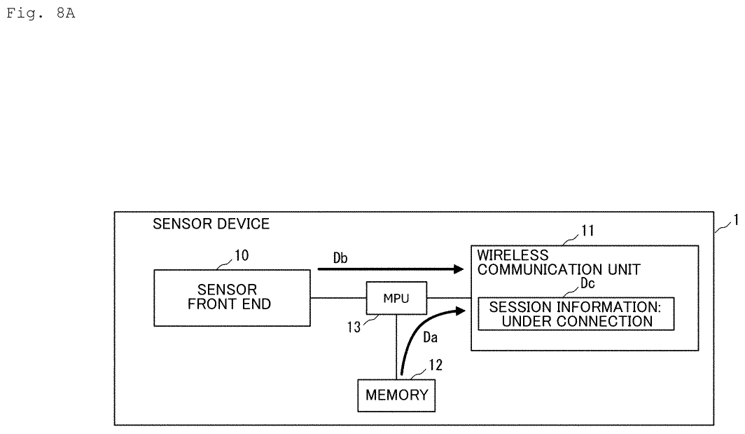

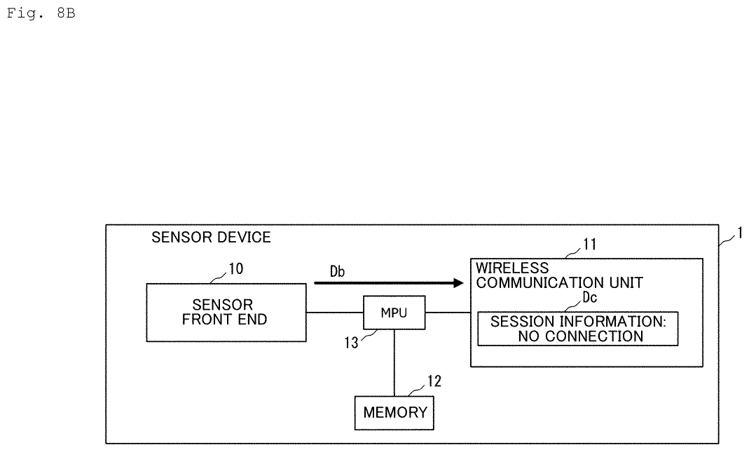

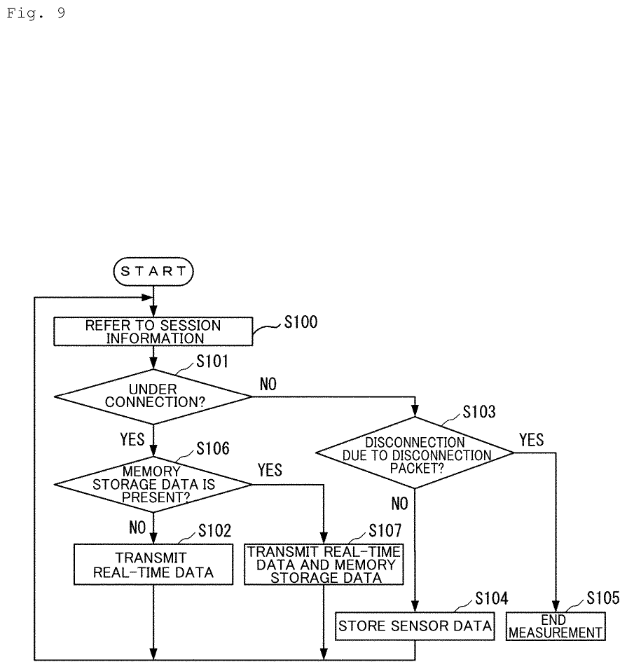

FIG. 8A and FIG. 8B are diagrams for explaining an operation of the sensor device 1 of the present example, and FIG. 9 is a flowchart for explaining an operation of the sensor device 1 of the present example. Processing of step S100 to step S105 in FIG. 9 is as described in the first example.

When the determination unit 130 determines to be under connection with the receiver 2 (YES in step S101 in FIG. 9) and the determination unit 130 determines that the sensor data Da which is not transmitted is stored in the memory 12 (YES in step S106 in FIG. 9), the data reading unit 134 of the sensor device 1 reads the sensor data Da stored in the memory 12. The data transfer processing unit 131 stores the sensor data Da read by the data reading unit 134 in the packet and passes the packet to the wireless communication unit 11. The wireless communication unit 11 wirelessly transmits the packet received from the data transfer processing unit 131 to the receiver 2 under connection.

When it is under connection with the receiver 2 and the sensor data Da which is not transmitted is stored in the memory 12, a packet in which the real-time data Db is stored is output to the wireless communication unit 11, and a packet in which the sensor data Da read from the memory 12 is stored is output to the wireless communication unit 11, as shown in FIG. 8A. The wireless communication unit 11 can alternately transmit the packet in which the real-time data Db is stored and the packet in which the sensor data Da read from the memory 12 is stored or simultaneously transmit the packets in parallel by transmitting in a prescribed rule not causing delay in transmission of the real-time data Db (step S107 in FIG. 9). On the other hand, when it is under connection with the receiver 2 and the sensor data Da is not stored in the memory 12, only the packet in which the real-time data Db is stored is transmitted to the receiver 2 as described above (step S102 in FIG. 8B and FIG. 9).

A transmission format when the sensor data Da which is not transmitted is stored in the memory 12 is not limited to the example of the above-described step S107, and an arbitrary transmission format may be specified in advance. As will be described later, if a time stamp is given to the sensor data Da stored in the memory 12, the data can be rearranged in the order of time point on the reception side.

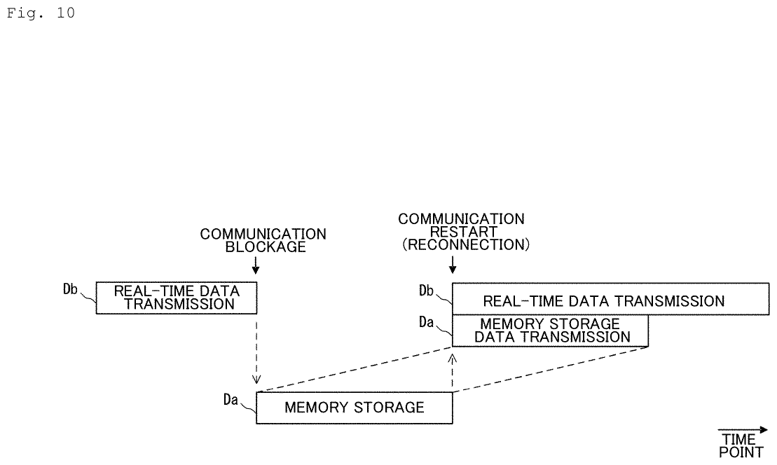

As described above, the sensor device 1 of the present example can read the sensor data Da from the memory 12 in parallel with the real-time data Db at appropriate timing during the measurement. For example, in the example shown in FIG. 10, when the sensor device 1 and the receiver 2 are reconnected and the communication is resumed, the sensor data Da is read from the memory 12 and transmitted to the receiver 2 together with the real-time data Db.

In the present example, since the reading operation can be automatically completed without making the user conscious of the reading of the data from the sensor device 1, the convenience for the user can be improved. In addition, in the present example, since free capacity of the memory 12 can be automatically increased, the time required for maintaining the data missing preventing function can be significantly extended.

### Third Example

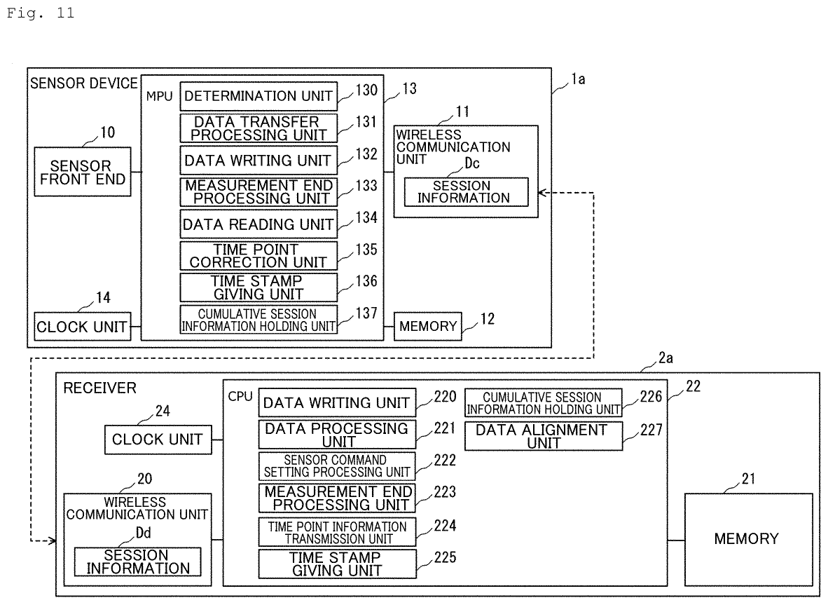

Next, a third example of the present invention will be described. The present example is an example of a more convenient way to organize and sort sensor data with respect to the first example. FIG. 11 is a block diagram showing a configuration of a sensor data collection system according to the present example. The sensor data collection system of the present example is configured by a sensor device 1a and a receiver 2a.

The sensor device 1a is obtained by adding a clock unit 14 to the sensor device 1 described in the first example, and further adding a time point correction unit 135, a time stamp giving unit 136, and a cumulative session information holding unit 137 as functions realized by the MPU 13.

The receiver 2a is obtained by adding a clock unit 24 to the receiver 2 described in the first example, and further adding a time point information transmission unit 224, a time stamp giving unit 225, a cumulative session information holding unit 226, and a data alignment unit 227 as functions realized by the CPU 22.

The sensor device 1a and the receiver 2a includes the clock units 14 and 24 with high accuracy called a real-time clock. In addition, the cumulative session information holding unit 137 of the sensor device 1a can count the number of times of connection from the first connection to the receiver 2a, and holds cumulative session information indicating what number of times of connection has been made. Similarly, the cumulative session information holding unit 226 of the receiver 2a can count the number of times of connection from the first connection to the sensor device 1a, and holds cumulative session information. For example, when the communication is blocked due to deterioration of the radio wave condition or the like and then a reconnection is performed, the number of times of connection is added by 1.

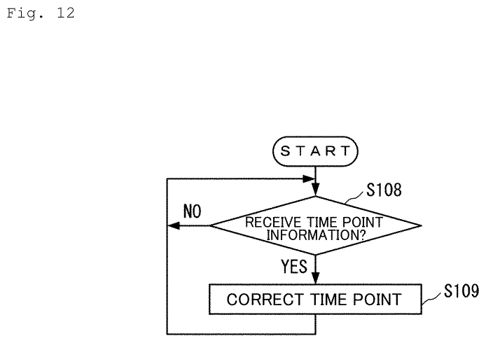

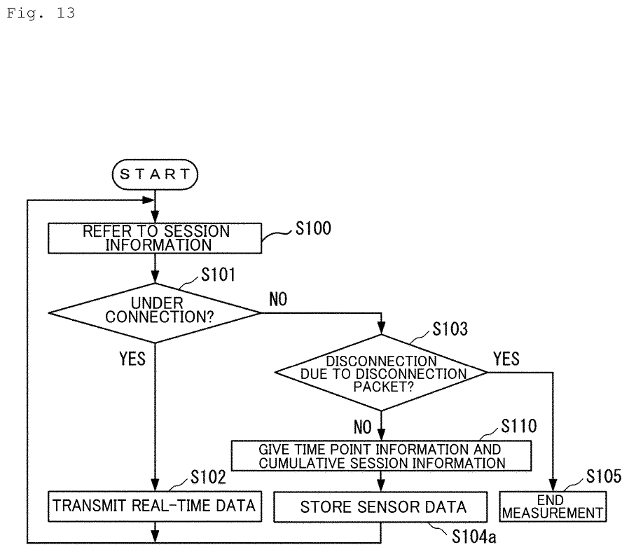

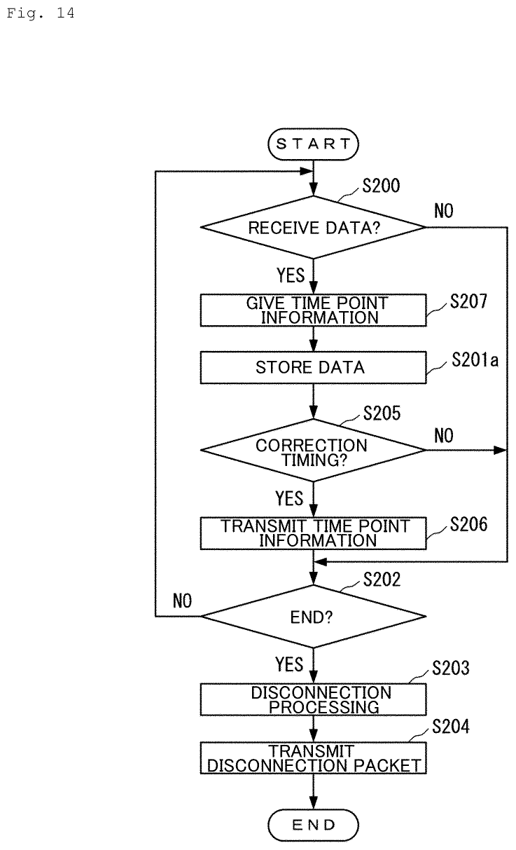

FIG. 12 and FIG. 13 are flowcharts for explaining an operation of the sensor device 1a, and FIG. 14 is a flowchart for explaining an operation of the receiver 2a.

When receiving a data packet in which the time point information is stored from the receiver 2a under connection (YES in step S108 in FIG. 12), the data transfer processing unit 131 of the sensor device 1a takes out the time point information from the received data packet and passes it to the time point correction unit 135. The time point correction unit 135 corrects the time point being measured by the clock unit 14 based on the time point information received from the data transfer processing unit 131 (step S109 in FIG. 12).

The processing of step S100 to step S103 and step S105 in FIG. 13 is as described in the first example.

When the determination unit 130 determines that the communication with the receiver 2a is disconnected (NO in step S101 in FIG. 13) and the determination unit 130 determines that the communication is not disconnected due to disconnection packet reception from the receiver 2a (NO in step S103 in FIG. 13), the time stamp giving unit 136 of the sensor device 1a acquires the time point information from the clock unit 14 and acquires the cumulative session information from the cumulative session information holding unit 137, and gives the time point information and the cumulative session information to the sensor data Da acquired from the sensor front end 10 (step S110 in FIG. 13). Specifically, the time stamp giving unit 136 stores the time point information and the cumulative session information in a header part of sensor data Da, for example.

The data writing unit 132 of the sensor device 1a stores the sensor data Da to which the time point information and the cumulative session information are given in the memory 12 (step S104a in FIG. 13). Other operations are the same as those of the sensor device 1 described in the first example.

Next, an operation of the receiver 2a will be described. The processing of step S202 to step S204 in FIG. 14 is as described in the first example. When prescribed correction timing comes (YES in step S205 in FIG. 14), the time point information transmission unit 224 of the receiver 2a acquires the time point information from the clock unit 24, stores it in the packet, and passes it to the wireless communication unit 20. The wireless communication unit 20 wirelessly transmits the packet received from the time point information transmission unit 224 to the sensor device 1a under connection (step S206 in FIG. 14).

By doing this, in the present example, the time point of the sensor device 1a and the receiver 2a can be synchronized. The correction timings for the time point include when the sensor device 1a and receiver 2a are connected for the first time, when they are reconnected, and at regular intervals, for example.

Note that although the sensor command setting processing unit 222 and the time point information transmission unit 224 are separately described in the present example, the sensor command setting processing unit 222 and the time point information transmission unit 224 may be combined into one unit.

When receiving the data packet in which the real-time data Db is stored from the sensor device 1a (YES in step S200 in FIG. 14), the time stamp giving unit 225 of the receiver 2a extracts the real-time data Db from the received data packet. The time stamp giving unit 225 acquires the time point information from the clock unit 24 and gives the time point information to the real-time data Db (step S207 in FIG. 14). Specifically, the time stamp giving unit 225 stores the time point information in the header part of the real-time data Db, for example. The data writing unit 220 stores the real-time data Db to which the time point information is given in the memory 21 (step S201a in FIG. 14).

Note that the time stamp giving unit 225 and the data writing unit 220 do not give (update) the time point information for memory storage data temporarily stored in the memory 12 of the sensor device 1a, that is, sensor data Da to which the time point information and the cumulative session information are already given, and stores the data in the memory 21.

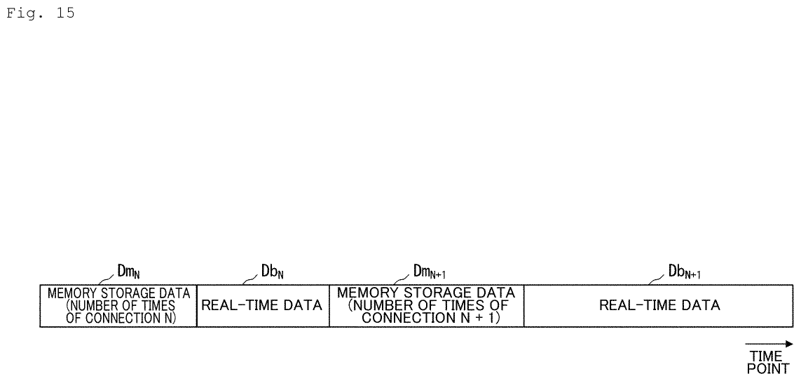

According to the present example, the data can be aligned as shown in FIG. 15. However, the data alignment unit 227 described below is not an essential component of the present invention, and may be provided in a place other than the receiver 2a.

The data alignment unit 227 rearranges the real-time data received from the sensor device 1a and stored in the memory 21 and the memory storage data read out after temporarily stored in the memory 12 of the sensor device 1a and stored in the memory 21 in the order of time point.

Specifically, the data alignment unit 227 separates the memory storage data temporarily stored in the memory 12 of the sensor device 1a among the data stored in the memory 21 based on the cumulative session information. For example, when the number of times of connection indicated by the cumulative session information changes from N (N is a positive integer) to N+1, it is possible to search for a break between the memory storage data indicating the number of times of connection N by the cumulative session information and the memory storage data indicating the number of times of connection N+1 by the cumulative session information. By doing this, the memory storage data can be separated by integrating the memory storage data indicating the same number of times of connection by the cumulative session information into one.

Then, the data alignment unit 227 connects the memory storage data and the real-time data so that the real-time data at the immediately after time point follows behind the data at the slowest time point among the memory storage data indicating the number of times of connection N by the cumulative session information. Further, the data alignment unit 227 searches for the real-time data at the slowest time point from the real-time data at the time point before the data at the earliest time point among the memory storage data indicating the number of times of connection N+1 by the cumulative session information. Then, the data alignment unit 227 connects the real-time data and the memory storage data so that the memory storage data indicating the number of times of connection N+1 by the cumulative session information follows behind the searched real-time data.

In the example shown in FIG. 15, a group of the memory storage data indicating the number of times of connection N by the cumulative session information is defined as Dm_{N}, and a group of the memory storage data indicating the number of times of connection N+1 by the cumulative session information is defined as Dm_{N+1}. In addition, a group of the real-time data is defined as Db_{N}, in which a head is connected to the data at the slowest time point among the memory storage data Dm_{N} and an end is connected to the data of the earliest time point among the memory storage data Dm_{N+1}. Further, a group of the real-time data in which the head is connected to the data at the slowest time point among the memory storage data Dm_{N+1} is defined as Db_{N+1}.

As described above, in the present example, the time point synchronization between the sensor device 1a and the receiver 2a is periodically performed. By doing this, since the time point information given to the memory storage data stored in the memory 12 of the sensor device 1a and the time point information given to the real-time data by the receiver 2a are matched, the data can be easily combined. Further, in the present example, since the cumulative session information reflecting the session information of the wireless communication is given to the memory storage data, the cumulative session information and the time point information can be used as an index for rearranging the sensor data, and data complementation (data linkage) can be performed more reliably. In the present example, since the time point order deviation does not occur in the sensor data, a data set which is very easily utilized by the user can be provided.

Although the example in which the present example is applied to the first example is described in FIG. 12 to FIG. 14, it is needless to say that the present example may be applied to the second example.

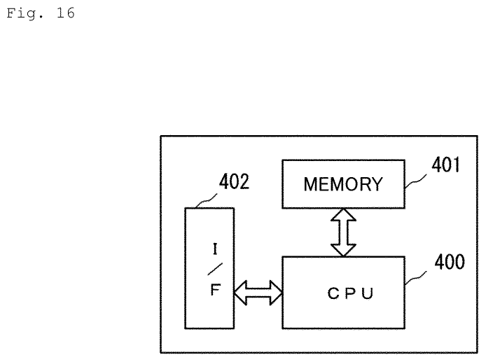

The data writing unit 220, the data processing unit 221, the time point information transmission unit 224, the time stamp giving unit 225, the data alignment unit 227, and the wireless communication unit 20 of the receivers 2 and 2a described in the first example to the third example can be realized by a computer including a CPU, a memory, and an interface and a program for controlling these hardware resources. FIG. 16 shows a configuration example of this computer.

The computer includes a CPU 400, a memory 401, and an interface device (I/F) 402. The hardware of the clock unit 24, the wireless communication unit 20, or the like are connected to the I/F 402. The CPU 400 (CPU 22) of the receivers 2 and 2a executes the processing described in the first example to the third example in accordance with a sensor data collection program stored in the memory 401 (memory 21).

The determination unit 130, the data transfer processing unit 131, the data writing unit 132, the measurement end processing unit 133, the data reading unit 134, the time point correction unit 135, the time stamp giving unit 136, the cumulative session information holding unit 137, and the wireless communication unit 11 of the sensor devices 1 and 1a can also be realized by the computer. The hardware of the sensor front end 10, the clock unit 14, and the wireless communication unit 11 or the like are connected to the I/F 402 of the sensor devices 1 and 1a. The CPU 400 (MPU 13) of the sensor devices 1 and 1a executes the processing described in the first example to the third example in accordance with the sensor data collection program stored in the memory 401 (memory 12).

In such a computer as described above, the sensor data collection program for realizing the sensor data collection method of the present invention is provided in a state of being recorded on a recording medium such as a flexible disc, a CD-ROM, a DVD-ROM, and a memory card. In addition, the program can also be provided via the network.

Some or all of the above-described examples are also described in the following supplements, but are not limited to the following.

(Supplement 1) A sensor data collection method includes a first step of, by a sensor device, referring to session information managed by a wireless communication unit of the sensor device and determining whether or not to be under connection with a receiver, a second step of, by the sensor device, stopping storing sensor data output from a sensor front end of the sensor device in real-time in a memory of the sensor device and wirelessly transmitting the sensor data to the receiver when determining to be under connection with the receiver based on the session information, and a third step of, by the sensor device, storing the sensor data in the memory when determining that communication with the receiver is disconnected based on the session information and determining that the communication is not disconnected due to disconnection packet reception from the receiver.

(Supplement 2) In the sensor data collection method described in Supplement 1, the second step includes a step of wirelessly transmitting the sensor data output from the sensor front end in real-time and the sensor data read from the memory to the receiver when determining to be under connection with the receiver and determining that the sensor data which is not transmitted is stored in the memory, and wirelessly transmitting the sensor data output from the sensor front end in real-time to the receiver when determining to be under connection with the receiver and determining that the sensor data which is not transmitted is not stored in the memory.

(Supplement 3) In the sensor data collection method described in Supplement 1 or 2, the third step includes a step of, by an MPU of the sensor device, acquiring time point information from a clock unit of the sensor device, giving cumulative session information indicating the number of times of connection with the receiver and the time point information which are held by the MPU to the sensor data output from the sensor front end, and storing the sensor data to which the time point information and the cumulative session information are given in the memory when determining that the communication with the receiver is disconnected and determining that the communication is not disconnected due to the disconnection packet reception from the receiver.

(Supplement 4) The sensor data collection method described in Supplement 3 further includes a fourth step of, by the sensor device, receiving the time point information periodically transmitted from the receiver under connection and a fifth step of, by the sensor device, correcting the time point being measured by the clock unit of the sensor device based on the time point information received from the receiver.

(Supplement 5) A sensor device of the present invention includes a sensor front end configured to output sensor data indicating measured physical amounts, a wireless communication unit configured to communicate with a receiver, a memory for storing the sensor data, and an MPU configured to stop storing the sensor data in the memory and wirelessly transmit the sensor data to the receiver in real-time via the wireless communication unit when determining to be under connection with the receiver based on session information managed by the wireless communication unit and store the sensor data in the memory when determining that communication with the receiver is disconnected based on the session information and determining that the communication is not disconnected due to disconnection packet reception from the receiver.

(Supplement 6) In the sensor device described in Supplement 5, the MPU wirelessly transmits the sensor data output from the sensor front end in real-time and the sensor data read from the memory to the receiver when determining to be under connection with the receiver and determining that the sensor data which is not transmitted is stored in the memory, and wirelessly transmits the sensor data output from the sensor front end in real-time to the receiver when determining to be under connection with the receiver and determining that the sensor data which is not transmitted is not stored in the memory.

(Supplement 7) The sensor device described in Supplement 5 or 6 further includes a clock unit configured to measure time, and the MPU holds cumulative session information indicating the number of times of connection with the receiver, and acquires time point information from the clock unit, gives the time point information and the cumulative session information to the sensor data output from the sensor front end, and stores the sensor data to which the time point information and the cumulative session information are given in the memory when determining that the communication with the receiver is disconnected and determining that the communication is not disconnected due to the disconnection packet reception from the receiver.

(Supplement 8) In the sensor device described in Supplement 7, the MPU corrects time point being measured by the clock unit based on the time point information received from the receiver by the wireless communication unit.

### INDUSTRIAL APPLICABILITY

The present invention can be applied to a technique for collecting the sensor data from the sensor device.

### REFERENCE SIGNS LIST

1, 1a Sensor device
2,2a Receiver
10 Sensor front end
11, 20 Wireless communication unit
12, 21 Memory
13 MPU
22 CPU
100 Sensor circuit
101 Analog front end circuit
102 AD converter
130 Determination unit
131 Data transfer processing unit
132, 220 Data writing unit
133, 223 Measurement end processing unit
134 Data reading unit
135 Time point correction unit
136, 225 Time stamp giving unit
137, 226 Cumulative session information holding unit
221 Data processing unit
222 Sensor command setting processing unit
224 Time point information transmission unit
227 Data alignment unit

1-8. (canceled)

9. A sensor data collection method by a sensor device including a sensor front end configured to output sensor data indicating measured physical amounts; a wireless communication circuit configured to communicate with a receiver; a memory for storing the sensor data; and a micro processing unit (MPU), the method comprising:
referring to session information managed by the wireless communication circuit and determining whether or not the sensor device is under connection with the receiver;
stopping storing sensor data output from the sensor front end in real-time in the memory and wirelessly transmitting the sensor data to the receiver when it is determined based on the session information that the sensor device is under connection with the receiver; and
storing the sensor data in the memory when it is determined based on the session information that communication with the receiver is disconnected and that the communication is not disconnected due to disconnection packet reception from the receiver.

10. The sensor data collection method according to claim 9, wherein
when it is determined based on the session information that the sensor device is under connection with the receiver, further wirelessly transmitting the sensor data output from the sensor front end in real-time and the sensor data read from the memory to the receiver if the sensor data which is not transmitted is stored in the memory, and further wirelessly transmitting the sensor data output from the sensor front end in real-time to the receiver if the sensor data which is not transmitted is not stored in the memory.

11. The sensor data collection method according to claim 9, wherein
the sensor device further includes a clock circuit configured to measure time,
when storing the sensor data in the memory, the MPU acquires time point information from the clock circuit, gives cumulative session information indicating the number of times of connection with the receiver and the time point information which are held by the MPU to the sensor data output from the sensor front end, and stores the sensor data to which the time point information and the cumulative session information are given in the memory if it is determined that the communication with the receiver is disconnected but the communication is not disconnected due to the disconnection packet reception from the receiver.

12. The sensor data collection method according to claim 11, further comprising:
receiving the time point information periodically transmitted from the receiver under connection; and
correcting the time point being measured by the clock circuit based on the time point information received from the receiver.

13. A sensor device comprising:
a sensor front end configured to output sensor data indicating measured physical amounts;
a wireless communication circuit configured to communicate with a receiver;
a memory for storing the sensor data; and
a micro processing unit (MPU) configured to:
refer to session information managed by the wireless communication circuit and determine whether or not the sensor device is under connection with the receiver;
stop storing the sensor data in the memory and wirelessly transmit the sensor data to the receiver in real-time via the wireless communication circuit when it is determined based on the session information managed by the wireless communication circuit that the sensor device is under connection with the receiver; and
store the sensor data in the memory when it is determined based on the session information that communication with the receiver is disconnected but that the communication is not disconnected due to disconnection packet reception from the receiver.

14. The sensor device according to claim 13, wherein
when it is determined that the sensor device is under connection with the receiver, the MPU further wirelessly transmits the sensor data output from the sensor front end in real-time and the sensor data read from the memory to the receiver if the sensor data which is not transmitted is stored in the memory, and further wirelessly transmits the sensor data output from the sensor front end in real-time to the receiver if the sensor data which is not transmitted is not stored in the memory.

15. The sensor device according to claim 13, further comprising:
a clock circuit configured to measure time, wherein
the MPU holds cumulative session information indicating the number of times of connection with the receiver, and acquires time point information from the clock circuit, gives the time point information and the cumulative session information to the sensor data output from the sensor front end, and stores the sensor data to which the time point information and the cumulative session information are given in the memory if it is determined that the communication with the receiver is disconnected but the communication is not disconnected due to the disconnection packet reception from the receiver.

16. The sensor device according to claim 15, wherein
the MPU corrects a time point being measured by the clock circuit based on the time point information received from the receiver by the wireless communication circuit.

17. The sensor data collection method according to claim 10, wherein
the sensor device further includes a clock circuit configured to measure time,
when storing the sensor data in the memory, the MPU acquires time point information from the clock circuit, gives cumulative session information indicating the number of times of connection with the receiver and the time point information which are held by the MPU to the sensor data output from the sensor front end, and stores the sensor data to which the time point information and the cumulative session information are given in the memory if it is determined that the communication with the receiver is disconnected but the communication is not disconnected due to the disconnection packet reception from the receiver.

18. The sensor data collection method according to claim 17, further comprising:
receiving the time point information periodically transmitted from the receiver under connection; and
correcting the time point being measured by the clock circuit based on the time point information received from the receiver.

19. The sensor device according to claim 14, further comprising:
a clock circuit configured to measure time, wherein
the MPU holds cumulative session information indicating the number of times of connection with the receiver, and acquires time point information from the clock circuit, gives the time point information and the cumulative session information to the sensor data output from the sensor front end, and stores the sensor data to which the time point information and the cumulative session information are given in the memory if it is determined that the communication with the receiver is disconnected but the communication is not disconnected due to the disconnection packet reception from the receiver.

20. The sensor device according to claim 19, wherein
the MPU corrects a time point being measured by the clock circuit based on the time point information received from the receiver by the wireless communication circuit.

21. A sensor data collection method comprising:
referring, by a sensor device, to session information managed by a wireless communication unit of the sensor device;
determining, by the sensor device, whether the sensor device is under connection with a receiver based on the session information;
wirelessly transmitting, by the sensor device, sensor data output from a sensor front end of the sensor device to the receiver in real-time when it is determined that the sensor device is under connection with the receiver;
storing, by the sensor device, the sensor data in a memory of the sensor device when it is determined that communication with the receiver is disconnected and that the communication is not disconnected due to disconnection packet reception from the receiver; and
stopping, by the sensor device, storing of the sensor data in the memory when wirelessly transmitting the sensor data to the receiver.

22. The sensor data collection method according to claim 21, further comprising:
wirelessly transmitting, by the sensor device, the sensor data output from the sensor front end in real-time and sensor data read from the memory to the receiver when it is determined that the sensor device is under connection with the receiver and sensor data which is not transmitted is stored in the memory.

23. The sensor data collection method according to claim 21, wherein the sensor device includes a clock circuit configured to measure time, the method further comprising:
acquiring, by the sensor device, time point information from the clock circuit;
giving, by the sensor device, cumulative session information indicating the number of times of connection with the receiver and the time point information to the sensor data output from the sensor front end; and
storing, by the sensor device, the sensor data to which the time point information and the cumulative session information are given in the memory when it is determined that the communication with the receiver is disconnected but the communication is not disconnected due to the disconnection packet reception from the receiver.

24. The sensor data collection method according to claim 23, further comprising:
receiving, by the sensor device, time point information periodically transmitted from the receiver when under connection; and
correcting, by the sensor device, the time point being measured by the clock circuit based on the time point information received from the receiver.

25. The sensor data collection method according to claim 21, further comprising:
setting, by the sensor device, a measurement operation end state when it is determined that the communication with the receiver is disconnected due to the disconnection packet reception from the receiver, wherein in the measurement operation end state, transmission of sensor data to the receiver and storage of sensor data in the memory are not performed.
